(19) United States
(12) Patent Application Publication
(10) Pub. No.: US 2021/0106228 A1
(43) Pub. Date: Apr. 15, 2021

(54) Systems and Methods for Wireless Monitoring of Patient Parameters and Initiating Mitigations Based on Quality of Service (QoS) Measurements

(71) Applicant: Physio-Control, Inc., Redmond, WA (US)

(72) Inventors: Clinton T. Siedenburg, Everett, WA (US); Daniel W. Piraino, Seattle, WA (US); Ray Sharif, Lake Forest Park, WA (US); Cathlene Durand Buchanan, Shoreline, WA (US)

(21) Appl. No.: 17/066,523

(22) Filed: Oct. 9, 2020

Related U.S. Application Data
(60) Provisional application No. 62/913,923, filed on Oct. 11, 2019.

(51) Int. Cl. A61B 5/00

(52) U.S. Cl. CPC A61B 5/0024; A61B 5/0004; A61B 5/0036; A61B 5/7292; A61B 5/7475; A61B 5/002; A61B 5/746

(57) ABSTRACT

An example system for wireless monitoring of patient parameters and initiating mitigations based on quality of service (QoS) measurements includes a plurality of sensors connected to a patient, a therapy module, and a monitor module that executes instructions to receive the signals from the plurality of sensors over the wireless communication link, determine therapy commands for delivering therapy to the patient and send the therapy commands to the therapy module, based on the QoS measurement being below a threshold initiate a short-term mitigation that includes providing an alert and to determine a modification to the therapy commands, based on the QoS measurement continuing to remain below the threshold, subsequently initiate a long-term mitigation that includes employing a secondary communication technique between the plurality of sensors and the monitor module to replace the wireless communication link, and controlling the therapy module according to the therapy commands during the long-term mitigation.

### CROSS-REFERENCE TO RELATED APPLICATION

The present application claims priority to U.S. provisional application No. 62/913,923, filed on Oct. 11, 2019, the entire contents of which are herein incorporated by reference.

### BACKGROUND

In the medical industry, monitors are provided to capture, process, display, archive, and review vital signs of a patient. Defibrillators deliver therapy such as defibrillation of ventricular fibrillation, pacing, and synchronized cardioversion. Monitors often are in communication with defibrillators to provide information or instructions to deliver therapy. Defibrillator-monitors and monitor-defibrillators perform both functions.

To capture vital signs of a patient, the monitors include sensing technology and are connected to the patient by wires or by hoses. The monitors also include a display screen, user interface, etc., and the wires can be in a form that connects to specific sensing technology (e.g., electrocardiogram (ECG), pulse oximetry (SpO₂), etc.). Other example sensing technology provided by the monitor includes (e.g., invasive blood pressure (IP), temperature, SpMet, SpCO, airway pressure, airway flow, etc.) each of which may require a wired connection to the patient for detection of vital signs. The monitors can also have connections for hoses, such as tubes for non-invasive blood pressure (NIBP) or side stream access to intubated patients for capnography (etCO₂).

It is desirable to eliminate the need for cables and hoses connecting patient sensors to monitors or defibrillator-monitor devices so as to make the monitor devices more portable and adaptable to other use cases, and to make it much easier to move a patient from the field to a hospital.

### SUMMARY

Within examples described herein, methods and systems for wireless monitoring of patient parameters and initiating mitigations based on quality of service (QoS) measurements are described.

Within additional examples described herein, methods and systems for wireless monitoring of patient parameters and processing of received vital sign measurements are described.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 illustrates a system for wireless monitoring of patient parameters and initiating mitigations based on quality of service (QoS) measurements, according to an example implementation.

FIG. 2 illustrates a block diagram of an example sensor of the plurality of sensors, according to an example implementation.

FIG. 3 illustrates a block diagram of an example of the monitor module, according to an example implementation.

FIG. 4 illustrates a block diagram of an example of the therapy module, according to an example implementation.

FIG. 5 illustrates an example communication of data from the plurality of sensors to the monitor module, according to an example implementation.

FIG. 6 illustrates an example communication of data from the plurality of sensors to the therapy module, according to an example implementation.

FIG. 7 shows a flowchart of an example of a method for wireless monitoring of patient parameters and initiating mitigations based on quality of service (QoS) measurements, according to an example implementation.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

Within examples, methods and systems for wireless monitoring of patient parameters and initiating mitigations based on quality of service (QoS) measurements are described. Wireless monitoring of patient parameters occurs by sensing and at least partially processing physiological parameters at the patient so that physiological signals may be digitized to allow wireless transmission of the data or data stream to a defibrillator-monitor, monitor-defibrillator, monitor, or defibrillator for further processing, display, use, or archival. Individual patient sensors are positioned to collect data, and then wirelessly transmit collected data to the external monitor. A wireless communication link is chosen to accommodate a bandwidth of the signal transmitted. More fully processed waveforms (e.g., SpO₂, SpMet, SpCO, etCO₂, airway pressure, ECG, etc.) or low data rate signals (e.g., systolic/diastolic blood pressure numbers, internal temperature) can be reliably transmitted using wireless transmission protocols such as Wi-Fi, Bluetooth, Bluetooth Low Energy (BLE), WiGig, ZigBee, Z-Wave, 2G, 3G, 4G, 5G, LTE, etc. Less processed waveforms, such as raw analog-to-digital converted (ADC) data from sensors or multi-lead electrocardiogram (ECG) may utilize a custom interface for reliable, low latency wireless transmission (e.g., proprietary wireless transmission protocols in the available Wi-Fi, Bluetooth, or medical bands) in place of the established wireless protocols such as Wi-Fi, Bluetooth, Bluetooth Low Energy (BLE), WiGig, ZigBee, Z-Wave, 2G, 3G, 4G, 5G, LTE, etc. if needed.

By sensing vital signs at the patient, processing the analog vital sign data, utilizing wireless technology, and using a power source local to the sensor at the patient (e.g., battery power) cables and hoses between sensors and monitors may be removed. In some examples, sensors may be bundled to eliminate redundant power sources and processing components. For instance, using a bundled implementation, wires from electrodes and sensors that are positioned in various places on the patient are connected to a small module that wirelessly transmits collected data from the module to an untethered monitor.

Benefits with the example methods and systems described herein include (1) cleanability of sensors and devices especially as connectors are eliminated and especially when the sensors are single use, (2) lower cost as connectors and cables are eliminated from initial purchase and disposable sensors eliminate cleaning time, (3) ease of use when moving a patient especially one that is intubated, and (4) products may be tailored to customer needs and sensors may be added to existing product without a hardware upgrade as customer needs evolve.

For devices that must be reliable but operate wirelessly, mitigations are required under conditions in which wireless communication is temporarily unavailable. Examples described herein include short-term mitigations as well as long-term mitigations. Short-term mitigations include alerts, modifying the state or performance of the monitor and/or defibrillator, disabling particular functions, etc. Long-term mitigations include a secondary communication channel to replace the wireless communication channel between any two or more modules that communicate wirelessly. In one example, the monitor and defibrillator modules are brought in contact or near contact to each other allowing a different, highly reliable, short-range connection to be made between the two which could include an optical connection for a short-term or long-term mitigation. Alternatively, a physical connection could be made by direct electrical or optical contact between the two. A second alternative is to replace the wireless communication with a wired one. Similarly, when wireless communication is interrupted to a vital sign sensor, a secondary communication channel is provided to replace the wireless communication by bringing them into contact or near contact to each other.

In addition, a quality of service (QoS) of the wireless communication link is monitored and measured periodically or on a real time basis to detect the need for issuance of short-term or long-term mitigations. This may be done by adding time stamps to the data packets or other timing schemes as the data packets are sent, for example, from the sensor to the monitor, the monitor to the therapy (e.g., defibrillator) module, and back, or between any two modules in wireless communication. Furthermore, responses with time stamps (or other timing indicators) indicating time of reception may be returned to the sender. The monitor, for example, compares the time stamps being received from the sensor data stream to the time stamps internal to the monitor. Differences are computed and analyzed for a mean delay and deviations from the mean to assess a reliability and latency of the wireless channel. A QoS measure is then produced regarding the current reliability and latency of a channel. Based on the QoS, alerts may be presented to the operator and modes of operation may be affected, such as alerting the operator to engage a different communication means between the two, especially in delivering synchronized cardioversion. A similar alert mechanism may be provided on the sensor side. Based upon QoS, a wireless visual indicator may change color or state on each module affected.

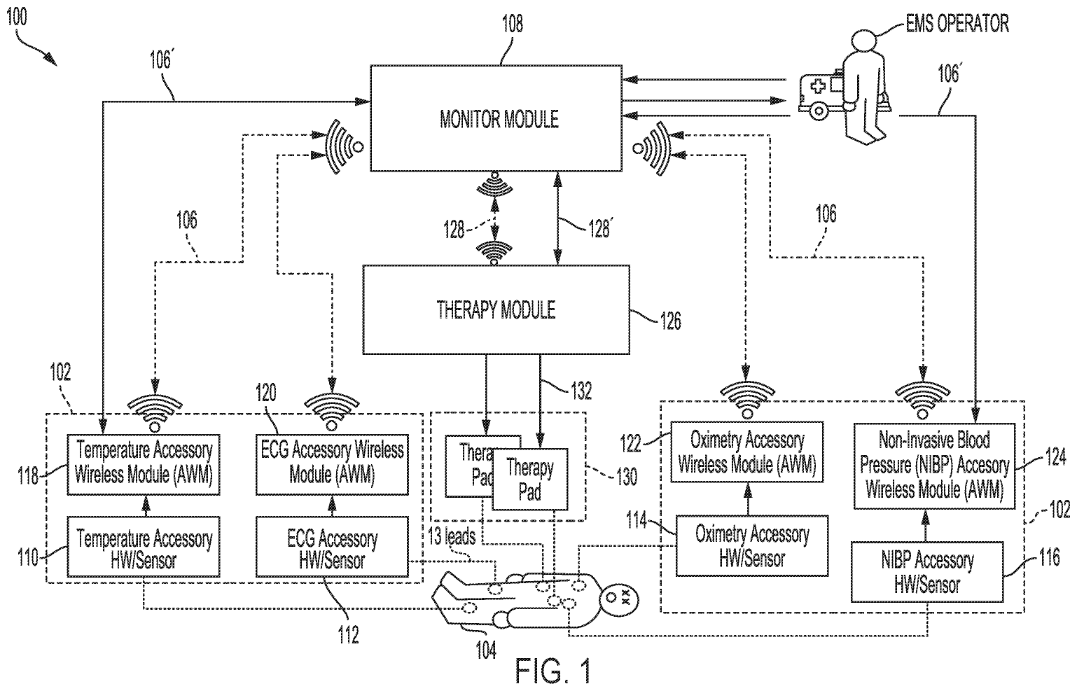

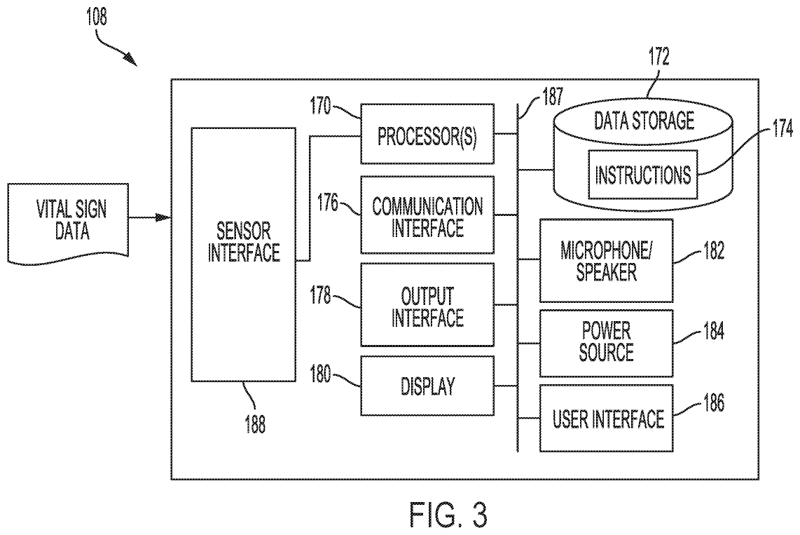

Referring now to the figures, FIG. 1 illustrates a system 100 for wireless monitoring of patient parameters and initiating mitigations based on quality of service (QoS) measurements, according to an example implementation. The system 100 includes a plurality of sensors 102, which may be positioned on or connected to a patient 104, to collect vital sign measurements of the patient 104 and to at least partially process the vital sign measurements to enable wireless transmission. The system 100 also includes a wireless communication link(s) 106, and a monitor module 108 with patient monitoring capability having a non-transitory computer-readable medium with a plurality of executable instructions stored therein and a processor adapted to execute the plurality of executable instructions (as shown in FIG. 3). The instructions are executable to receive the vital sign measurements from the plurality of sensors 102 over the wireless communication link 106, use the vital sign measurements to determine therapy commands for delivering therapy to the patient 104 and send the therapy commands to a therapy module 126, determine a quality of service (QoS) measurement of the wireless communication link 106, based on the QoS measurement being below a threshold, initiate a short-term mitigation that includes providing an alert and to determine a modification to the therapy commands, control the therapy module 126 according to the modification to the therapy commands during the short-term mitigation, based on the QoS measurement continuing to remain below the threshold, subsequently initiate a long-term mitigation that includes employing a secondary communication technique between the plurality of sensors 102 and the monitor module 108 to replace the wireless communication link 106, and control the therapy module 126 according to the therapy commands during the long-term mitigation.

Within examples, as shown in FIG. 1, the secondary communication technique is illustrated as implementing a secondary communication channel 106′ between the plurality of sensors 102 and the monitor module 108 and/or implementing a secondary communication channel 128′ between the monitor module and the therapy module 126. The secondary communication channel 106′ and/or the secondary communication channel 128′ can include another type of wireless communication, a wired communication, or an electromagnetic coupling, for example.

The plurality of sensors 102 may include any number or type of sensors, and some examples illustrated in FIG. 1 include a temperature sensor 110, an ECG sensor 112, an oximetry sensor 114, and a non-invasive blood pressure (NIBP) sensor 116. Each of the plurality of sensors 102 includes power source, such as batteries. Furthermore, a transmitter is coupled to each of the plurality of sensors 102 through a wired connection, and the transmitter wirelessly transmits the vital sign measurements to the monitor module 108. For example, as shown in FIG. 1, a temperature accessory wireless module (AWM) 118 is coupled to the temperature sensor 110, an ECG AWM 120 is coupled to the ECG sensor 112, an oximetry AWM 118 is coupled to the oximetry sensor 114, and an NIBP AWM 124 is coupled to the NIBP sensor 116. In some examples, respective AWMs of the sensors are incorporated within the sensor itself. Alternatively, more than one sensor can be bundled together into a housing and share an AWM, even different types of sensors.

It is noted that in some examples, one sensor may be used, or in other examples, one or sensors may be used (such as the plurality of sensors 102 shown in FIG. 1).

The plurality of sensors 102 measure the vital signs at the patient 104, process and wirelessly transmit partially processed measured data to the monitor module 108, or alternatively convey raw data without processing to the monitor module 108 instead. By measuring the vital signs at the patient 104, this eliminates the need for all hoses and cables between the patient 104 and the monitor module 108. Providing medical treatment to patients that are tethered to a device is fraught with difficulties, and thus, using the system 100 eliminates such problems. In addition, some existing sensors require hoses to connect to the monitor module 108, which necessitates the use of pumps internal to the monitor module 108 that drive size, weight, and heat of the monitor module 108 to increase. Hoses and cables require connectors that are often different for each vital sign requiring more connections than necessary if not all vital signs are used at once. Moreover, hoses, cables, and connectors must all be ruggedized since they are to last years under the harshest conditions. Ruggedization means larger volumes, increased weight, and much higher cost. Cables with higher weight deliver additional torque to the connector which means that it must be yet more rugged. As can be seen weight, size, and power capacity of the monitor module 108 can all be reduced by eliminating pumps in the monitor module 108.

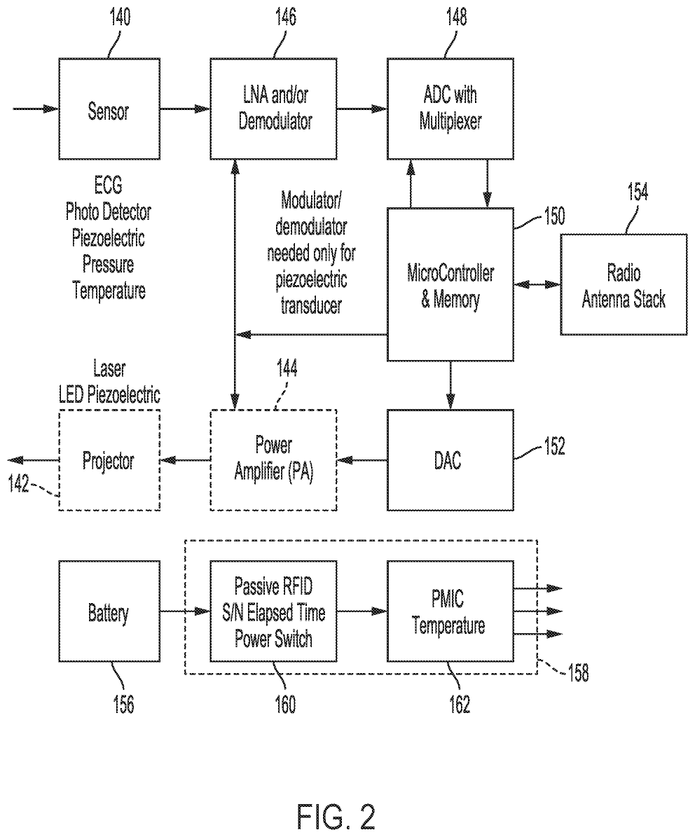

FIG. 2 illustrates a block diagram of an example sensor of the plurality of sensors 102, according to an example implementation. Each sensor has a sensor block 140 that contains a transducer to convert a particular vital sign to an electrical signal. For an ECG sensor, for example, it is a direct measurement of an electrical signal. For SpO2, SpCO, SpMet, etCO2, the sensor block 140 includes a photodetector. For airway pressure or invasive pressure sensors, the sensor block 140 is a pressure transducer. For a temperature sensor, the sensor block 140 is a temperature transducer. For ultrasound used in NIBP, the sensor block 140 may be a piezoelectric crystal. Some vital sign interrogation requires emission of energy referred to in FIG. 2 as a projector 142. Others such as ECG, temperature, and pressure do not require the projector 142 as the body produces the necessary energy itself. Thus, the projector 142 is an optional component of the sensor depending on a type of sensor. For SpO2, SpCO, SpMet, and etCO2, the projector 142 includes an array of LEDs of various wavelengths. For ultrasound versions of NIBP, the same piezoelectric sensor may be used for transmission. For optical versions of NIBP, either an LED or laser is used. Where energy is emitted from the projector 142, there is a corresponding power amplifier (PA) 144.

The sensor also includes a low noise amplifier (LNA) 146. The LNA 146 has a high impedance for the ECG sensor, for example, and low impedance for other applications like a piezoelectric transducer, for example. The number of LNAs per sensor module varies. For example, the temperature sensor requires one, the airway sensor requires two or three, the blood gas sensors require two to six, and the ECG sensor requires two to fifteen. For ultrasound that operates in the MHz range, there may also be a demodulator to mix the low bandwidth signal down to baseband before being sent to the ADC.

The output of the LNA 146 is received by the ADC/multiplexer block 148. The number of LNA channels varies from 1 to 15, for example, and so an input analog multiplexer (MUX) is provided to accept up to 16 inputs, for example. A microcontroller 150 selects which MUX input is scheduled for conversion. The analog-to-digital converter (ADC) converts the data and feeds data to the microcontroller 150 which places the data into a buffer. The microcontroller 150 also directs the digital-to-analog converter (DAC) 152 to output analog levels to the PA 144. The microcontroller 150 then sends data to a radio module 154 for transmission. The radio module 154, as described and shown in FIG. 1, may be a separate AWM module or may be included within the sensor.

The infrastructure to the sensor module also includes a battery 156 and power management integrated circuit (PMIC) 158 to supply the power at the correct voltages to the various parts of the sensor module. A passive RFID controls when the battery turns on by means of a switch 160. The microcontroller 150 commands the switch 160 to go off. The passive RFID switch 160 comes to life when energized by an external field and will return identification data such as a serial number and sensor type and status data such as battery energy level given the current temperature 162. The passive RFID switch 160 can be directed to connect the battery power to energize the microcontroller for operation. In this way, the battery energy can be preserved for only when the energy is to be used for sensing patient vital signs.

Further details of example types of the plurality of sensors 102 are described below.

In one example, the plurality of sensors 102 includes an ECG sensor. An ECG sensor may include various numbers of channels. Some examples are 1L, 3L, 6L, 12L, and 15L versions.

Another example includes an SpO2/SpCO/SpMet sensor. Such sensors use light of various and particular wavelengths at which gases have different absorption properties. By computing transmission and absorption ratios at these specially chosen wavelengths, values of SpO2, SpCO, SpMet, and others can be deduced. Since these are slowly varying signals, slower than ECG, all the same architecture can be used. Furthermore, the impedance of the signals is expected to be much lower so that a less complicated LNA than that used for ECG is required.

Another example includes an etCO2/airway pressure sensor. The etCO2 sensor uses the same technology as the SpO2/SpCO/SpMet sensor including LEDs and photodiodes. Airway pressure sensing may be added to the etCO2 sensor to obtain optimal measurement of ventilation rate, detection of incomplete chest recoil during CPR, identification of risk of pulmonary barotrauma, monitoring of beneficial intrathoracic pressure effects of LUCAS, avoidance of gastric insufflation risk during BVM ventilation, indication of incipient tension pneumothorax, and measurement and automatic documentation of PEEP.

Another example includes an NIBP sensor. There are two sensor approaches to NIBP; cuff-based and non-invasive, continuous blood pressure technology. In the reusable cuff-based system, however, there are just two numbers reported every ten minutes or so. In the cuff-based system, the pump is in the device attached to the patient, not the module/monitor. In the new non-invasive, continuous blood pressure technology, there is a steady stream of data.

Another example includes an invasive blood pressure sensor. Since it is invasive to an artery and sterile, it is disposable. In one example, the pressure sensor interfaces with a fluid line that extends from a bag of saline solution which can be pumped by hand with sufficient pressure to equalize to arterial pressure of the open-ended catheter inserted into the artery. Part way between the catheterized artery and the saline bag, the pressure transducer is in contact with the fluid. The pressure sensor has an electrical connection to which the limited-use sensor module connects.

Another example includes an invasive temperature sensor. The invasive temperature sensor assesses core temperature for body cooling purposes. It is invasive passing through the nasal cavity and down the throat or through the oral cavity and down the throat. Although it is not a rapidly changing signal, it is a continuous signal.

Another example includes a laryngoscope. The purpose of the laryngoscope is to assist with airway intubation and document intubation process for training purposes. Data rates are high for this video stream as compared to the vital signs discussed above. In the background, a time-stamped, compressed, video stream is communicated to the monitor module and archived along with the patient record.

Another example includes an ultrasound sensor. The purpose of ultrasound imaging is to perform a FAST exam where it can be determined if the heart is beating, if there is fluid around the heart preventing it from beating, or if blood is pooling indicating a life-threatening condition, for example. Another purpose for ultrasound is guidance for the catheterization process, needle guidance for aspirating fluid from around the heart, etc. Ultrasound imaging produces a lot of data and is a full high-speed video with frame rates as high as 60 Hz and higher and resolution, for example, at a minimum of 640×480 pixels, for example. It may be difficult to reliably wirelessly transmit data at that rate. Therefore, either processed acquisition data is transmitted, or compressed data is transferred, or both. Processed acquisition data is on the order of the same frame rate but resolutions of 128×400, about a factor of six less than the video frame. Therefore, the processed acquisition data can be sent to the monitor module and have the monitor format it to its own display. Alternatively, video clips could be saved within the ultrasound device and transferred in the background over a Wi-Fi connection, for example, so that the video clip is added to the patient record assembled in the monitor.

Another example sensor includes an accelerometer to provide cardio pulmonary resuscitation feedback. Accelerometers can be integrated into a disposable patch that is placed as a pad where the palms go for applying chest compressions. Outputs of the accelerometer can feed the processing of the therapy box algorithms and can be used to cue the appropriate voice prompts. Furthermore, the accelerometer signal would be sent to the monitor for processing, display, and archival with the patient record.

Referring back to FIG. 1, the wireless communication link 106 may be provided by the monitor module 108 and includes a wireless transmission device capable of providing a wireless channel according to a protocol (e.g., Bluetooth, Bluetooth Low Energy (BLE), WiGig, ZigBee, Z-Wave, 2G, 3G, 4G, 5G, and LTE, low latency custom, etc.), or contains multiple wireless transmission devices to be able to provide multiple wireless channels according to multiple protocols. The wireless communication link 106 may include multiple communication channels between each of the plurality of sensors 102 and the monitor module 108, and thus, may include multiple wireless communication links. The monitor module 108 selects one of the wireless communication protocols for use based on a type of the plurality of sensors 102 in use, for example, and an amount of data that is being transmitted. For example, more fully processed signals (e.g. SpO2, SpMet, SpCO, etCO2, airway pressure, ECG) or low data rate data (e.g. systolic/diastolic blood pressure numbers, internal temperature) can be reliably transmitted over BLE, for example. Less processed signals such as raw data from sensors, especially multi-lead ECG, may require a custom interface for reliable, low latency transmission or higher power off-the-shelf technology such as Wi-Fi, for example.

The monitor module 108 may be referred to as control and display module, and the system 100 is shown to further include the therapy module 126 that is in wireless communication over a second wireless communication link 128 with the monitor module 108. The therapy module 126 includes a defibrillator operable to deliver therapy to the patient 104 including one or more of defibrillation, pacing, and synchronized cardioversion according to the therapy instructions received from the monitor module 108. The therapy module 126 utilizes therapy pads 130 connected via wires 132 to the therapy module 126 for delivering therapy.

Thus, when it comes to applying therapy, a user may interact with the monitor module 108 to initiate a therapy (defibrillation, sync-cardio, pacing) command. That would be a high level command for transmission from the monitor module 108 to the therapy module 126 along with a selected ECG waveform for sync-cardio and pacing. The therapy module 126 would then run its own set of instructions to perform all the detailed execution of the high level command for execution of the therapy. Alternatively, the therapy command may include specific detailed instructions, such that the monitor module 108 controls the detailed execution of therapy by the therapy module 126.

In some examples, the monitor module 108 and the therapy module 126 can be a combined device, such that the monitor module 108 includes a defibrillator, and the second wireless communication link 128 is replaced with a hard-wired link. For a combined device, the monitor module 108 and the therapy module 126 can be included into one housing or can be under control by a common central processor, for example. In other examples, for a combined device, the monitor module 108 and the therapy module 126 can share components, such as a processor, power source, input/output ports, etc.

The system 100 enables vital sign measurements to be collected by the plurality of sensors 102 associated with the patient 104, and then the vital sign measurements are wirelessly transmitted to the monitor module 108. In some examples, the vital sign measurements are wirelessly transmitted to other devices, such as a server located at a hospital, a server located in the cloud, or another type of receiver in addition to or instead of the monitor module 108. Other types of devices that may be receivers in this example include a hospital bed controller that wirelessly receives outputs of sensor(s), such as a pressure sensor providing information indicative of whether the hospital bed is occupied, for example. Any type of receiver that is in wireless communication with the plurality of sensors 102 can benefit from the methods and systems described herein for QoS measurement and mitigation functionality.

Using the system 100 enables wireless sensing for advanced lifesaving (ALS) defibrillator/monitors to eliminate wires and hoses connecting sensors to the monitor module 108, and for controlling delivery of therapy based on wirelessly monitored signals. As described below, wirelessly sensed data is sent to the monitor module 108, which then communicates therapy commands wirelessly to the therapy module 126 that can deliver therapy to the patient through wired/connected therapy pads 130.

FIG. 3 illustrates a block diagram of an example of the monitor module 108, according to an example implementation. The monitor module 108 may take the form of a computing device with multiple storage partitions.

The monitor module 108 includes one or more processor(s) 170, and a non-transitory computer-readable medium (e.g., data storage) 172 storing instructions 174 that when executed by the one or more processor(s) 170 cause the one or more processor(s) 170 to perform functions of the monitor module 108.

To perform the functions, the monitor module 108 includes a communication interface 176, an output interface 178, a display 180, a microphone/speaker 182, a power source 184, and a user interface 186, and each component of the monitor module 108 is connected to a communication bus 187. The monitor module 108 also includes a sensor interface 188.

The communication interface 176 may be one or more wireless interfaces and one or more wireline interfaces that allow for both short-range communication and long-range communication to one or more networks or to one or more remote devices. Such wireless interfaces may provide for communication under one or more wireless communication protocols, Bluetooth, Wi-Fi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 176 may be configured to receive input data from one or more devices and may also be configured to send output data to other devices. The communication interface 176 thus may include hardware to enable communication between the monitor module 108 and other devices (not shown). The hardware may include transmitters, receivers, and antennas, for example. The communication interface 176 may also be capable of operating as a wireless access point.

The non-transitory data storage 172 may include or take the form of memory, such as one or more computer-readable storage media that can be read or accessed by the one or more processor(s) 170. The computer-readable storage media can include volatile and/or non-volatile storage components, such as optical, magnetic, organic or other memory or disc storage, which can be integrated in whole or in part with the one or more processor(s) 170. The non-transitory data storage 172 is considered non-transitory computer readable media. In some examples, the non-transitory data storage 172 can be implemented using a single physical device (e.g., one optical, magnetic, organic or other memory or disc storage unit), while in other examples, the non-transitory data storage 172 can be implemented using two or more physical devices.

The non-transitory data storage 172 thus is a computer readable medium, and instructions 174 are stored thereon. The instructions 174 include computer executable code.

The one or more processor(s) 170 may be general-purpose processors or special purpose processors (e.g., digital signal processors, application specific integrated circuits, etc.). The one or more processor(s) 170 may receive inputs from the communication interface 176 as well as from other sensors and process the inputs to generate outputs that are stored in the non-transitory data storage 172. The one or more processor(s) 170 can be configured to execute the instructions 174 (e.g., computer-readable program instructions) that are stored in the non-transitory data storage 172 and are executable to provide the functionality of the monitor module 108 described herein.

The output interface 178 outputs information for reporting or storage, and thus, the output interface 178 may be similar to the communication interface 176 and can be a wireless interface (e.g., transmitter) or a wired interface as well.

The display 180 includes a touchscreen or other type of display. The microphone/speaker 182 include capabilities to receive audio/voice instructions, and to output audio including audible prompts.

The power source 184 may include battery power, or a wired power means such as an AC power connection, or energy harvesting methods use light, motion, or heat.

The user interface 186 provides indicator LEDs for readiness status and power, to support failure analysis, operations, service, as well as software debugging. The user interface 186 may include Ethernet and USB ports as well as buttons.

The sensor interface 188 enables the plurality of sensors 102 to be magnetically attached, for example, to the monitor module 108 for storage. Each sensor module has a small magnet that will activate a Hall Effect sensor in the sensor interface 188 indicating the presence of a sensor module. When the arrival of a sensor module is detected, the monitor module 108 emits the field that activates the passive near-field communication (NFC) mechanism of the sensor and determines what type of sensor it is, queries its status such as power remaining, and preemptively pairs with the monitor module 108. When the Hall Effect sensor indicates that the sensor is being removed, the monitor module 108 activates the sensor module power through NFC. In this way, pairing of sensor to monitor will be unnoticed and sensor battery power will be conserved.

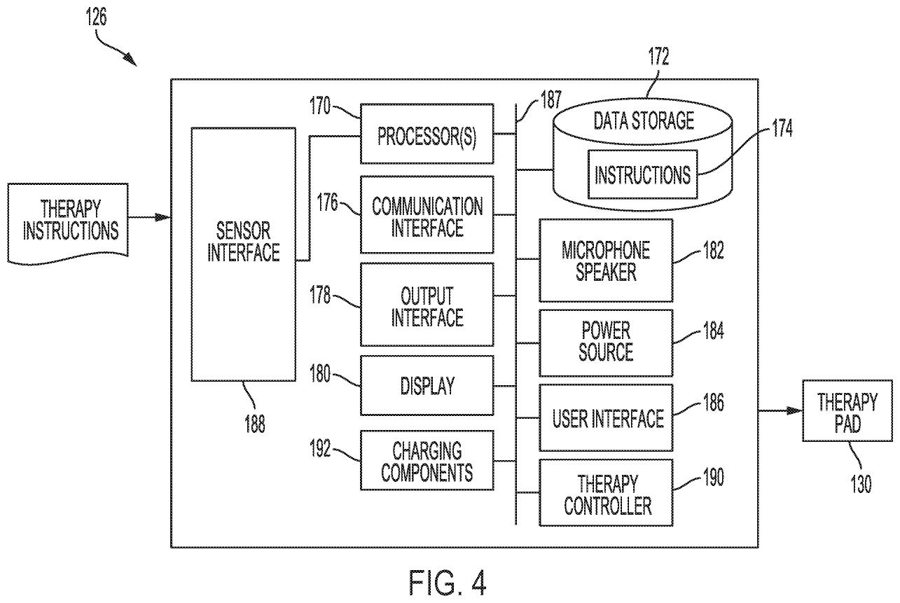

FIG. 4 illustrates a block diagram of an example of the therapy module 126, according to an example implementation. The therapy module 126 includes many components that are the same as included in the monitor module 108, and are referenced with the same reference numbers in FIGS. 3-4. Such components are not described again here.

The therapy module 126 also further includes a therapy controller 190 that connects to the therapy pads 130 and charging components 192. The therapy pads 130 are disposable and quickly connected/disconnected with a magnetic connection scheme, for example, to the sensor interface 188. Electrical connection is made to carry the high currents required for defibrillation. The ability to read identification over a serial bus is designed into the connection. The ECG and impedance data acquired by the therapy module 126 is sent to the monitor module 108 over the second wireless communication link 128 for display.

The therapy controller 190 may include a field programmable gate array (FPGA) state machine that receives high level commands from the processor 170. Such commands include charge to a certain energy level, shock, and discharge as well as setting a current level for pacing mode. The therapy controller 190 controls charging and discharging of an energy capacitor, detailed operation of the H-bridge, as well as the pacing circuitry that controls the current level within the charging components 192. A second FPGA state machine in the therapy controller 190 may be used to control analog-to-digital conversion of ECG and impedance waveforms so as to not burden the processor 170 with tight timing requirements. A circular buffer in the therapy controller 190 temporarily holds the data and alerts the processor 170 when another packet of predetermined size is available for transfer and processing.

Within examples, in instances where the monitor module 108 and the therapy module 126 are separate devices or separate components, the plurality of sensors 102 can be paired with the monitor module 108 and/or the therapy module 126.

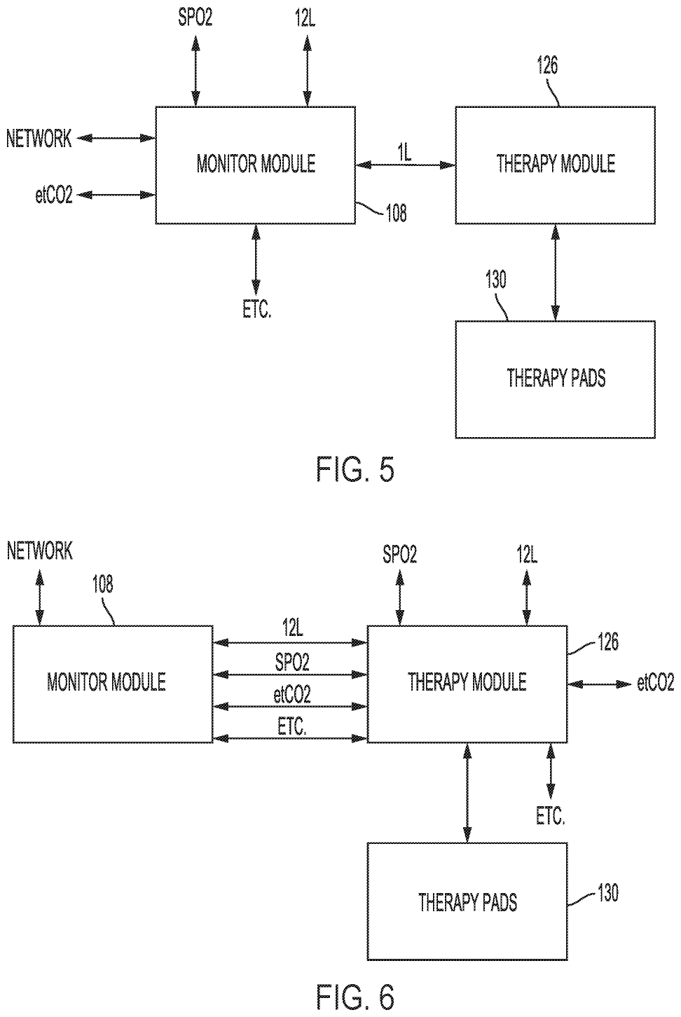

FIG. 5 illustrates an example communication of data from the plurality of sensors 102 (i.e., illustrated as SpO₂, 12L, and etCO₂ sensors, for example) to the monitor module 108, according to an example implementation. As shown in FIG. 5, the plurality of sensors 102 first send the outputs (digitized waveforms and/or raw vital sign data) to the monitor module 108 over wireless communication link(s) (such as, the wireless communication link(s) 106 shown in FIG. 1), and then the monitor module 108 processes the data to determine and wirelessly transmit therapy commands as well as any required waveform to the therapy module 126. Sending data to the monitor module 108 first may be an efficient way to move data around wirelessly among the modules.

FIG. 6 illustrates an example communication of data from the plurality of sensors 102 (i.e., illustrated as SpO₂, 12L, and etCO₂ sensors, for example) to the therapy module 126, according to an example implementation. As shown in FIG. 6, the wireless data flow requires exchange of larger data volumes between monitor module 108 and the therapy module 126 than that shown in FIG. 5. This is in part due to one lead of data between the monitor module 108 and the therapy module 126 in FIG. 5 and up to 15 leads of data plus other vital sign signals if needed between the monitor module 108 and the therapy module 126 in FIG. 6. Thus, in FIG. 6, the plurality of sensors 102 (i.e., illustrated as SpO₂, 12L, and etCO₂ sensors, for example) first send the outputs to the therapy module 126 over a wireless communication link (such as the wireless communication link 106 shown in FIG. 1), and then the therapy module 126 sends the data to the monitor module 108 over the second wireless communication link 128. The monitor module 108 processes the data to determine therapy commands and wirelessly transmit therapy commands and possibly vital sign signals back to the therapy module 126.

In some examples, data transmission may be performed so as to prioritize reception of data from certain sensors to thereby load balance the data. In an example where the sensors include ECG sensors, certain ECG data may be more important and can be prioritized for transmission and reception. A waveform may be clearest on lead V, for example, and thus such waveform data can be transmitted and received first while degrading transmission of other data (or transmitting at higher latencies), for example.

Within examples, as described above, the monitor module 108 and the therapy module 126 may be separate devices or combined in one device. In either example, whether the monitor module 108 is a stand-alone device or is combined with the therapy module 126, in operation, when the instructions 174 are executed by the one or more processor(s) 170 of the monitor module 108, the one or more processor(s) 170 is caused to perform functions including receive the vital sign measurements from the plurality of sensors 102 over the wireless communication link 106, use the vital sign measurements to determine therapy commands for delivering therapy to the patient and send the therapy commands to the therapy module 126, determine a quality of service (QoS) measurement of the wireless communication link 106, based on the QoS measurement being below a threshold, initiate a short-term mitigation that includes providing an alert and to determine a modification to the therapy commands, control the therapy module 126 according to the modification to the therapy commands during the short-term mitigation, based on the QoS measurement continuing to remain below the threshold, subsequently initiate a long-term mitigation that includes employing a secondary communication technique between the plurality of sensors 102 and the monitor module 108 to replace the wireless communication link, and control the therapy module 126 according to the therapy commands during the long-term mitigation.

The mitigations thus include short-term and long-term mitigations, and the short-term mitigation persists either until the long-term mitigation occurs or the QoS measurement improves. The short-term mitigation immediately occurs for safety reasons. The short-term mitigation persists indefinitely until either the operator established a secondary connection (e.g., a long-term mitigation) or the QoS improves and the short-term mitigation (and long-term mitigation) is no longer needed and the modes and features for therapy are restored. The long-term mitigations are a secondary link, wired or wireless, that replaces the primary wireless link. The short-term mitigation can alternatively be discontinued based on the long-term mitigation being initiated by the monitor module 108, such as the monitor module 108 initiated a secondary communication technique that is more reliable than the wireless communication link 106 (e.g., a more reliable wireless link or searching for another available wireless connection).

Within examples, the short-term mitigation can take many forms. Example short term mitigations include providing visual, audio, or tactile alerts to the user (e.g., to indicate to user to employ secondary communication techniques) based on low QoS measurements. Short term mitigations further include modifying user options or user interface behavior or system configuration (e.g., switching to non-demand pacing for a specific time duration, automatically switch over to AED mode), or disabling of certain features (e.g. disabling synchronized-cardioversion, demand pacing, etc.).

As one example, the short-term mitigation includes modifying operation of the defibrillator (the therapy module 126) to switch to non-demand pacing for a specific time duration. As another example, the short-term mitigation includes modifying operation of the defibrillator (the therapy module 126) to switch to an automated external defibrillator (AED) mode with the ability to control the AED functions using features on the therapy module 126. As another example, short term mitigation includes modifying operation of the defibrillator (the therapy module 126) to disable synchronized-cardioversion.

As a specific example, therapy may include synchronized cardioversion (sync-cardio), in which an R-wave detection algorithm is used that runs in the therapy module, and wireless ECG leads are used for vital sign detection. The vital sign detection senses a heart rhythm in order to properly time a delivery of a shock for atrial fibrillation therapy. In this case, the therapy module 126 would be analyzing the time stamps from sensor to monitor and then monitor to therapy module. If QoS is above a threshold, the therapy module 126 is armed to deliver sync-cardio therapy synchronized with the next R-wave. Prior to doing so, however, if the packet that contains the R-wave is found to have a time stamp or other timing indicator that is not expected, the therapy module 126 will instantly delay therapy until the next R-wave having an expected time stamp or timing indicator. In this way, sudden and/or temporary interruptions in the communication channel can be reliably caught so that adverse events will be avoided. A short-term mitigation thus disables synchronized cardio therapy, and a long-term mitigation (when wireless communication is not working with a QoS at or above the threshold) may be implemented connecting leads directly from the plurality of sensors 102 to the module.

The functions also include, based on the QoS measurement continuing to remain below the threshold for the duration of between about 60 ms to about 5s (e.g., depending on the type of therapy being provided and a timing of signals required to deliver the therapy), subsequently initiating a long-term mitigation that includes employing a secondary communication technique between the plurality of sensors 102 and the monitor module 108 to replace the wireless communication link 106. In one example, the secondary channel includes one of an electromagnetic coupling or a wired connection. In another example, the secondary channel includes an optical link. Still further, the secondary channel can include other short-range wireless communication links (e.g., such as Bluetooth low energy (BLE)).

In examples, where the monitor module 108 and the therapy module 126 are separate modules, the long-term mitigation can further include employing a further secondary channel between the monitor module and the therapy module. The secondary channel can include the secondary communication channel 128′ between the monitor module and the therapy module 126 shown in FIG. 1.

Since ECG is important to therapy delivery, a particular mitigation is provided for ECG in the event that the wireless communication QoS deteriorates. In such a case, the AWM of the sensor modules can be bypassed or removed from the disposable ECG wire harness and the wire harness can be attached directly to the monitor module 108. An electrical connection is made to the disposable wire harness and the analog signals are amplified and converted to a stream of data for however many channels are carried by the harness.

Since therapy delivery is so important, it too has a particular but different mitigation provided for when the normal wireless communication QoS breaks down between the monitor module 108 and the therapy module 126. This mitigation need not be an electrical connection as for ECG, but rather a secondary active link that works when the therapy module 126 and monitor module 108 are brought in close proximity for short-range communication. The short-range communication can be either of optical or electromagnetic technology.

Consequently, in instances in which wireless communication between the monitor module 108 and the therapy module 126, the monitor module 108 and the plurality of sensors 102, or the therapy module 126 and the plurality of sensors 102 is unavailable, deteriorates, or is simply below a QoS threshold, a long-term mitigation can include using short-range wireless communication techniques, such as electromagnetic coupling, to bring the two devices in contact or near contact to each other, or an optical communication, or switching to another secondary link (wired communication) (e.g., shown as the secondary communication channel 128′ in FIG. 1).

Many methods are possible to determine the QoS measurement. In one example, the monitor module 108 determines the QoS measurement of the wireless communication link(s) 106 (and/or of the second wireless communication link 128) by computing transmission delays based on time stamps of the vital sign measurements and time of receipt of the vital sign measurements, and based on the transmission delay, producing the QoS measurement indicative of reliability and latency of the wireless communication link.

Other examples for detection of the QoS measurement include use of time stamps or other timing indicators in the data stream, use of internal timing information of the wireless receiving device, algorithms to compare time information of received data to internal timing information and determine if there is a sufficient match, “pinging” to measure round trip timing measurements from one device to another and back, statistical analysis of data reception times based upon an internal clock to aid in determining reliability of future timing performance, and statistical analysis of number of re-tries to send data to aid in determining reliability of future timing performance.

Thus, the QoS of the wireless communication link(s) 106 is measured on a real time basis, such as by adding time stamps to the data packets as they are sent from the plurality of sensors 102 (added by the sensors) to the monitor module 108, the monitor module 108 to the therapy module 126, and back. Furthermore, responses with time stamps indicating time of reception are returned to the sender. The monitor module 108, for example, compares the time stamps being received from the sensor data stream to the time stamps internal to monitor module 108. The differences are computed and analyzed for the mean delay and the deviations from the mean to assess the reliability and latency of the channel. A QoS measure is then produced regarding the current reliability and latency of a channel. In this way, sudden and temporary interruptions in the communication channel can be reliably caught so that adverse events will be avoided.

For different types of therapy being provided, different mitigations can be implemented. In an example where the therapy includes synchronized cardioversion in which the plurality of sensors 102 include wireless electrocardiogram (ECG) leads, the therapy module 126 analyzes time stamps in the therapy commands for determination of a second QoS measurement of the second wireless communication link 128, and based on the second QoS measurement satisfying the threshold, the therapy module 126 delivers sync-cardio therapy according to the therapy instructions. However, based on the second QoS measurement not satisfying the threshold, the therapy module 126 delays therapy until the second QoS measurement satisfies the threshold. Synch cardioversion requires 60 millisecond timing from the heart's R wave to delivery of therapy to the heart. In the system shown in FIG. 1, this includes data transmission across the first wireless communication link 106 and the second wireless communication link 128. The 60 milliseconds timing includes processing times as well and so a time allowance for wireless latency is very small, such as on the order of 8 milliseconds for the two hops across the first wireless communication link 106 and the second wireless communication link 128. As a result, a QoS of both the first wireless communication link 106 and the second wireless communication link 128 can be verified, and when either is below a threshold, the mitigations can be implemented.

Thus, a short-term mitigation includes disabling sync cardio therapy since it cannot be reliably implemented with the timing requirements needed. A long-term second mitigation can include bringing the monitor module 108 and the therapy module 126 together for a different and more reliable secondary communication such as near field communication for example (no wires/connectors: electromagnetic, optical, etc.).

In an example where the therapy includes pacing of the patient 104, normal operation includes R-wave detection algorithms implemented for pacing therapy running in the therapy module 126 and all leads of ECG data are wirelessly transmitted to the monitor module 108. When low QoS is detected for the wireless communication links 106 and/or 128, the monitor module 108 and the therapy module 126 alert user through voice prompts and/or LEDs or any other visual, audible, or haptic indicator. A short-term mitigation includes the therapy module 126 continuing pacing automatically switching to non-demand pacing for a specified time period (e.g., 10 minutes) until QoS recovers. A long-term mitigation includes bringing the monitor module 108 and the therapy module 126 together for a different and more reliable secondary communication such as near field communication.

In an example where the therapy includes manual defibrillation operation, mitigation for low QoS detected for the first wireless communication link 106 and the second wireless communication link 128 includes the monitor module 108 and the therapy module 126 alerting the user through voice prompts and/or LEDs. In an example of a short-term mitigation including system mode changes, the monitor module 108 providing audio prompts and visual alerts indicating to follow lighted steps of the therapy module 126, and the therapy module 126 switching to AED mode. The therapy module 126 then may use lighted LEDs and pictures and flashing buttons or other displays of information to give instructions and allow the operator to control the defibrillation by controls on the therapy module 126. A long-term mitigation includes bringing the monitor module 108 and the therapy module 126 together for a different and more reliable secondary communication such as near field communication.

The different types of therapy that are provided have different tolerances for acceptable latency of the wireless communication link 106. The amount of latency acceptable can control a wireless protocol that is used. Such wireless protocols can include proprietary protocols that monitor QoS of the wireless link, for example.

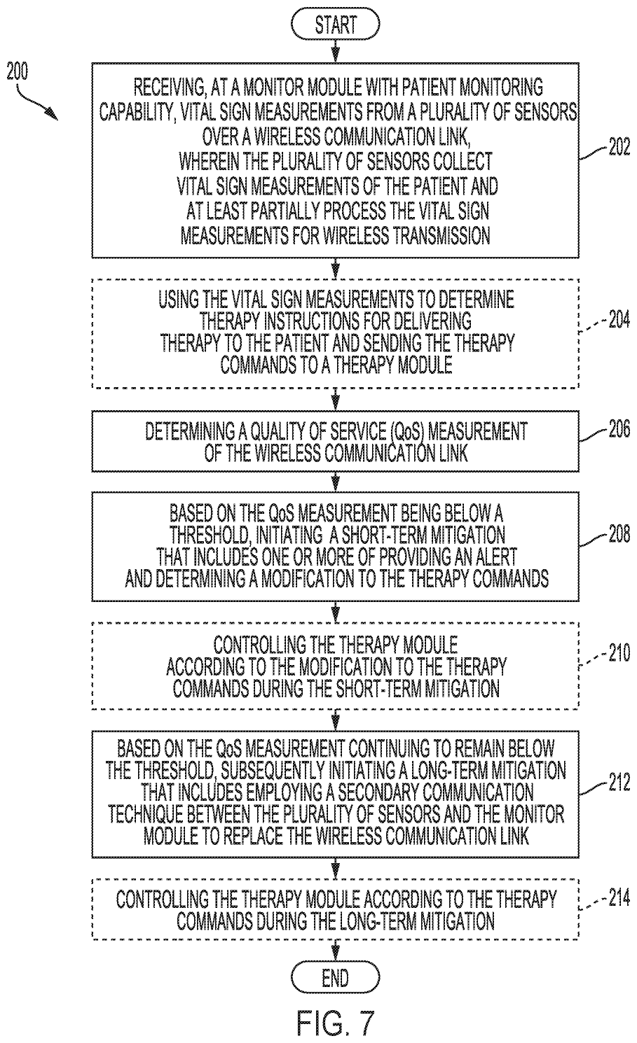

FIG. 7 shows a flowchart of an example of a method 200 for wireless monitoring of patient parameters and initiating mitigations based on quality of service (QoS) measurements, according to an example implementation. Method 200 shown in FIG. 7 presents an example of a method that could be used with the system 100 shown in FIG. 1, for example. Further, devices or systems may be used or configured to perform logical functions presented in FIG. 7. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 200 may include one or more operations, functions, or actions as illustrated by one or more of blocks 202-214. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long-term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 7, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 202, the method 200 includes receiving, at the monitor module 108 with patient monitoring capability, vital sign measurements from the plurality of sensors 102 over the wireless communication link(s) 106. The plurality of sensors 102 can be connected to the patient 104 to collect vital sign measurements of the patient 104 and to at least partially process the vital sign measurements for wireless transmission. The vital sign measurements received at the monitor module 108 can include or be in the form of digitized waveforms (as processed and prepared by the plurality of sensors 102), analog waveforms, or digital data including numerical values of temperature or a blood pressure reading as output by the plurality of sensors, for example.

In some examples, block 202 includes receiving, at the monitor module 108 with patient monitoring capability, one or more vital sign measurements from one or more sensors of the plurality of sensors 102 over the wireless communication link(s) 106. Thus, not all sensors of the plurality of sensors 102 may provide outputs or vital sign measurements at all times or less than all of the plurality of sensors 102 (e.g., only one sensor) may be in communication with the monitor module 108 at a given time.

At block 204, the method 200 optionally includes using the vital sign measurements to determine therapy instructions for delivering therapy to the patient 104 and sending the therapy commands and/or waveform data to a therapy module. This can include the combined monitor module 108 and therapy module 126 referencing stored algorithms to determine an appropriate therapy to deliver based on the collected vital signs.

In some examples, block 204 more simply includes generating therapy commands for delivering therapy to the patient and sending the therapy commands to the therapy module. The therapy commands can be generated programmatically by the monitor module 108 using the vital sign measurements to determine appropriate therapy for the patient. In other examples, the therapy commands can be generated based on user input, and the monitor module 108 is executes instructions for prompting a user for input including the therapy commands. The user may view the vital sign measurements and provide a selection of a command on a display of the monitor module 108, or provide other input indicating the therapy command.

In further examples, the monitor module 108 includes a control and display module, and the method 200 further comprises the control and display module wirelessly transmitting the therapy commands, and waveforms as needed, to the therapy module 126 over the second wireless communication link 128. Functionality of the monitor module 108 is to process and display vital sign signals and to be the primary user interface. The functionality of the therapy module 126 is to perform all therapy functionality, including automatic shock decisions, based upon its own secondary user interface or a high level message to do so initiated by the user from the monitor module 108 primary interface.

At block 206, the method 200 includes determining a quality of service (QoS) measurement of the wireless communication link 106.

At block 208, the method 200 includes based on the QoS measurement being below a threshold, initiating a short-term mitigation that includes one or more of providing an alert and determining a modification to the therapy commands.

In one example, the alert is provided (in form or audio/visual notification, for example) to provide information indicating that a wireless signal strength is low and to inform a user to connect a sensor to the monitor module 108, for example, such as by bringing the monitor module 108 in proximity to the sensor for electromagnetic coupling. The alert may alternatively inform a user that certain therapy is no longer available, such as sync cardio, which requires very precise timing.

In other examples, the monitor module 108 determines the modification to the therapy commands to provide instructions for reduced functionality of the therapy module 126 for safety concerns, for example. A modification can include transitioning from demand pacing to non-demand pacing as a result of an unreliable receipt of ECG data from sensors.

In still other examples, both an alert is provided and the modification to the therapy command is determined.

The QoS measurement threshold may be different and is based on the therapy being provided as well as the timing requirements needed. An example threshold metric may include latency, which refers to time taken for the data packet to reach a destination. Values on the order of a few milliseconds may be considered acceptable QoS.

Another threshold metric may include packet delay variation, which is a delay specified from a start of the data packet being transmitted at a source: to the start of the data packet being received at a destination. If data packets are transmitted every 10 ms, and a second data packet is received 20 ms after the first data packet, the packet delay variation is 10 ms, and such values nay be considered below an acceptable threshold depending on the therapy being provided.

Another threshold metric may be data packet loss, and when data packet loss is more than 5%, the QoS measurement may be considered below an acceptable threshold.

Within examples, the monitor module 108 includes a defibrillator (e.g., the therapy module 126) operable to deliver therapy to the patient including one or more of defibrillation, pacing, and synchronized cardioversion according to the therapy instructions, and initiating the short-term mitigation further includes modifying operation of the defibrillator to switch to non-demand pacing for a specific time duration. In addition, or alternatively, initiating the short-term mitigation further includes modifying operation of the defibrillator to switch to an automated external defibrillator (AED) mode. Moreover, in addition or alternatively, initiating the short-term mitigation further includes modifying operation of the defibrillator to disable synchronized-cardioversion.

At block 210, the method 200 optionally includes controlling the therapy module 126 according to the modification to the therapy commands during the short-term mitigation. In this manner, the therapy module 126 is controlled to have limited or modified functionality during the low QoS measurement time period. This functionality persists during the short-term mitigation. The functions of block 210 are performed in examples in which the short-term mitigation actions include the monitor module 108 determining the modifications to the therapy commands. Otherwise, the monitor module 108 would continue controlling the therapy module according to the original therapy commands initially determined.

At block 212, the method 200 includes based on the QoS measurement continuing to remain below the threshold, subsequently initiating a long-term mitigation that includes employing a secondary communication technique between the plurality of sensors 102 and the monitor module 108 to replace the wireless communication link 106. Initiating the long-term mitigation can include the monitor module 108 attempting to establish a secondary communication channel over electromagnetic coupling or other wireless technique different than in use for the first wireless communication channel 106, for example. The monitor module 108 can attempt to establish the secondary communication channel by determining presence of the sensors in proximity for electromagnetic coupling, for example.

At block 214, the method 200 optionally includes controlling the therapy module 126 according to the therapy commands during the long-term mitigation. Thus, once the long-term mitigation has been implemented, the control of the therapy module 126 can return to the original therapy commands for full functionality.

In further examples, the wireless communication link 106 is provided by the monitor module 108 and includes one of standard or custom communications (e.g., proprietary wireless transmission protocols in the available Wi-Fi, Bluetooth, or medical bands or established wireless protocols such as Wi-Fi, Bluetooth, Bluetooth Low Energy (BLE), WiGig, ZigBee, Z-Wave, 2G, 3G, 4G, 5G, LTE), and the method 200 further comprises the monitor module 108 selecting one of standard or custom communications for use based on a type of the plurality of sensors 102 in use.

In some further examples, the method 200 is modified to be performed by the monitor module 108 such that there is not a distinction between a short-term and long-term mitigation. Rather, the monitor module 108 receives the vital sign measurements from the one or more sensors over the wireless communication link, generates therapy commands for delivering therapy to the patient, determines a quality of service (QoS) measurement of the wireless communication link, and based on the QoS measurement being below a threshold, initiates a mitigation that includes one or more of (i) providing an alert, (ii) determining a modification to the therapy commands, and (iii) employing a secondary communication technique between the one or more sensors and the monitor module 108 to replace the wireless communication link. Thus, once the QoS falls below a predetermined metric, the monitor module 108 programmatically attempts to mitigate any problems by executing one of the three options, or by executing more than one of the three options simultaneously.

As an example, when the QoS is below the threshold, the monitor module 108 can provide an alert to the user and employ the secondary communication technique between the one or more sensors and the monitor module to replace the wireless communication link by employing an electromagnetic coupling communication.

In this example, the monitor module 108 and the therapy module 126 can be a combined device, such that the monitor module 108 includes a defibrillator, and the second wireless communication link 128 is replaced with a hard-wired link. Alternatively, the monitor module 108 can perform the modified method and provide outputs to the therapy module 126.

The systems and methods described herein are very beneficial to solve the cable management problem by measuring vital signs at the patient, wirelessly transmitting the vital sign data away from the patient, and using disposable sensors or portions of sensors (such as pre-packaged ECG cables) just once wherever possible. Clean-ability is greatly improved by removing the need for connectors from normal operation for all but ECG mitigations. The reusable portions of ECG connectors used for mitigations are low-profile to be easily cleaned and any hard to clean part is disposable. Clean-ability is also greatly improved through the use of inductive charging of internal batteries, non-mechanical buttons, and surfaces void of sharp inner angles.

Implementations of this disclosure provide technological improvements that are particular to computer technology, for example, those concerning vital sign data collection and processing. Computer-specific technological problems, such as low QoS communication channel occurrences, can be wholly or partially solved by implementations of this disclosure. For example, implementation of this disclosure allows for mitigation of problems that would otherwise occur due to lack of the wireless communication channel by creating alternative communication channels so that therapy can continue to be provided to the patient. Implementations of this disclosure can thus introduce new and efficient improvements in the ways in which data is collected at the patient and transmitted to the monitor module 108 for therapy commands to be generated even when the wireless communication channel may be experiencing problems.

By the term “substantially” and “about” used herein, it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination or any sub-combination, and all of such possibilities are intended to be within the scope of the disclosure.

The description of the different advantageous arrangements has been presented for purposes of illustration and description and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to best explain the principles of the examples, the practical application, and to enable others of ordinary skill in the art to understand the disclosure for various examples with various modifications as are suited to the particular use contemplated.

What is claimed is:

1. A system for wireless monitoring of patient parameters and initiating mitigations based on quality of service (QoS) measurements, the system comprising:
a plurality of sensors to collect vital sign measurements of a patient and to at least partially process the vital sign measurements for wireless transmission;
a wireless communication link;
a therapy module with a defibrillator capability;
a monitor module with patient monitoring capability in communication with the therapy module, the monitor module having a non-transitory computer-readable medium with a plurality of executable instructions stored therein and a processor adapted to execute the plurality of executable instructions to:
receive the vital sign measurements from the plurality of sensors over the wireless communication link;
generate therapy commands for delivering therapy to the patient and send the therapy commands to the therapy module;
determine a quality of service (QoS) measurement of the wireless communication link;
based on the QoS measurement being below a threshold, initiate a short-term mitigation that includes one or more of providing an alert and determining a modification to the therapy commands;
based on the QoS measurement continuing to remain below the threshold, subsequently initiate a long-term mitigation that includes employing a secondary communication technique between the plurality of sensors and the monitor module to replace the wireless communication link; and
control the therapy module according to the therapy commands during the long-term mitigation.

2. The system of claim 1, wherein the short-term mitigation includes determining the modification to the therapy commands and the processor of the monitor module is further adapted to execute the plurality of executable instructions to:
control the therapy module according to the modification to the therapy commands during the short-term mitigation.

3. The system of claim 1, wherein generate therapy commands for delivering therapy to the patient comprises the processor of the monitor module executing the plurality of executable instructions to:
use the vital sign measurements to determine therapy commands for delivering therapy to the patient.

4. The system of claim 1, wherein generate therapy commands for delivering therapy to the patient comprises the processor of the monitor module executing the plurality of executable instructions to:
prompt a user for input including the therapy commands.

5. The system of claim 1, wherein the wireless communication link is provided by the monitor module and includes one of Bluetooth or Wi-Fi communications, and wherein the monitor module selects one of the Bluetooth or Wi-Fi communications for use based on a type of the plurality of sensors in use.

6. The system of claim 1, wherein the monitor module and the therapy module are a combined device which comprises a defibrillator operable to deliver therapy to the patient including one or more of defibrillation, pacing, and synchronized cardioversion according to the therapy commands.

7. The system of claim 6, wherein the short-term mitigation includes modifying operation of the defibrillator to switch to non-demand pacing for a specific time duration.

8. The system of claim 6, wherein the short-term mitigation includes modifying operation of the defibrillator to switch to an automated external defibrillator (AED) mode.

9. The system of claim 6, wherein the short-term mitigation includes modifying operation of the defibrillator to disable synchronized-cardioversion.

10. The system of claim 1, wherein the monitor module includes a control and display module, and the therapy module is a separate component from the monitor module, wherein the therapy module is operable to deliver therapy to the patient including one or more of defibrillation, pacing, and synchronized cardioversion according to the therapy commands, wherein the therapy module is in wireless communication over a second wireless communication link with the control and display module to receive the therapy commands.

11. The system of claim 10, wherein the long-term mitigation further includes employing a further secondary channel between the monitor module and the therapy module.

12. The system of claim 10, wherein the therapy includes synchronized cardioversion in which the plurality of sensors include wireless electrocardiogram (ECG) leads, and wherein
the therapy module analyzes time stamps in the therapy commands for determination of a second QoS measurement of the second wireless communication link,
based on the second QoS measurement satisfying the threshold, the therapy module delivers sync-cardio therapy according to the therapy commands; and
based on the second QoS measurement not satisfying the threshold, the therapy module delays therapy until the second QoS measurement satisfies the threshold.

13. The system of claim 1, wherein the plurality of sensors include power sources.

14. The system of claim 1, wherein the secondary communication technique includes a short-range wireless communication.

15. The system of claim 1, wherein the secondary communication technique includes a wired connection.

16. The system of claim 1, further comprising:
a transmitter coupled to each of the plurality of sensors through a wired connection, wherein the transmitter wirelessly transmits the vital sign measurements to the module.

17. The system of claim 1, wherein the monitor module determines the quality of service (QoS) measurement of the wireless communication link by:
computing transmission delays based on time stamps of the vital sign measurements and time of receipt of the vital sign measurements; and
based on the transmission delays, producing the QoS measurement indicative of reliability and latency of the wireless communication link.

18. A non-transitory computer-readable medium having stored therein a plurality of executable instructions, which when executed by a monitor module having a processor causes the monitor module to perform functions comprising:
receiving vital sign measurements from a plurality of sensors over a wireless communication link, wherein the plurality of sensors collect vital sign measurements of a patient and at least partially process the vital sign measurements for wireless transmission;
generating therapy commands for delivering therapy to the patient and sending the therapy commands to a therapy module;
determining a quality of service (QoS) measurement of the wireless communication link;
based on the QoS measurement being below a threshold, initiating a short-term mitigation that includes one or more of providing an alert and determining a modification to the therapy commands;
based on the QoS measurement continuing to remain below the threshold, subsequently initiating a long-term mitigation that includes employing a secondary communication technique between the plurality of sensors and the monitor module to replace the wireless communication link; and
controlling the therapy module according to the therapy commands during the long-term mitigation.

19. The non-transitory computer-readable medium of claim 18, wherein the wireless communication link includes one of Bluetooth or Wi-Fi communications, and the functions further comprise:
selecting one of the Bluetooth or Wi-Fi communications for use based on a type of the plurality of sensors in use.

20. The non-transitory computer-readable medium of claim 18, wherein the monitor module and the therapy module are a combined device which comprises a defibrillator operable to deliver therapy to the patient including one or more of defibrillation, pacing, and synchronized cardioversion according to the therapy commands, and
wherein initiating the short-term mitigation includes modifying operation of the defibrillator to switch to non-demand pacing for a specific time duration.

21. The non-transitory computer-readable medium of claim 18, wherein the monitor module and the therapy module are a combined device which comprises a defibrillator operable to deliver therapy to the patient including one or more of defibrillation, pacing, and synchronized cardioversion according to the therapy commands, and
wherein initiating the short-term mitigation includes modifying operation of the defibrillator to switch to an automated external defibrillator (AED) mode.

22. The non-transitory computer-readable medium of claim 18, wherein the monitor module and the therapy module are a combined device which comprises a defibrillator operable to deliver therapy to the patient including one or more of defibrillation, pacing, and synchronized cardioversion according to the therapy commands, and
wherein initiating the short-term mitigation includes modifying operation of the defibrillator to disable synchronized-cardioversion.

23. The non-transitory computer-readable medium of claim 18, wherein the monitor module includes a control and display module and the therapy module is a separate component from the monitor module, and the functions further comprise:
the control and display module wirelessly transmitting the therapy commands to a therapy module over a second wireless communication link.

24. The non-transitory computer-readable medium of claim 23, wherein initiating the long-term mitigation further includes employing a further secondary channel between the monitor module and the therapy module.

25. The non-transitory computer-readable medium of claim 23, wherein the therapy includes synchronized cardioversion in which the plurality of sensors include wireless electrocardiogram (ECG) leads, and
wherein initiating the long-term mitigation further includes:
analyzing time stamps in the therapy commands for determination of a second QoS measurement of the second wireless communication link,
based on the second QoS measurement satisfying the threshold, delivering sync-cardio therapy according to the therapy commands; and
based on the second QoS measurement not satisfying the threshold, delaying therapy until the second QoS measurement satisfies the threshold.

26. The non-transitory computer-readable medium of claim 18, wherein determining the quality of service (QoS) measurement of the wireless communication link comprises:
computing a transmission delay based on time stamps of the vital sign measurements and time of receipt of the vital sign measurements; and
based on the transmission delay, producing the QoS measurement indicative of reliability and latency of the wireless communication link.

27. A system for wireless monitoring of patient parameters and initiating mitigations based on quality of service (QoS) measurements, the system comprising:
one or more sensors to collect vital sign measurements of a patient and to at least partially process the vital sign measurements for wireless transmission;
a wireless communication link;
a monitor module with patient monitoring capability, the monitor module having a non-transitory computer-readable medium with a plurality of executable instructions stored therein and a processor adapted to execute the plurality of executable instructions to:
receive the vital sign measurements from the one or more sensors over the wireless communication link;
generate therapy commands for delivering therapy to the patient;
determine a quality of service (QoS) measurement of the wireless communication link; and
based on the QoS measurement being below a threshold, initiate a mitigation that includes one or more of (i) providing an alert, (ii) determining a modification to the therapy commands, and (iii) employing a secondary communication technique between the one or more sensors and the monitor module to replace the wireless communication link.

28. The system of claim 27, wherein the wireless communication link is provided by the monitor module and includes one of Bluetooth or Wi-Fi communications, and the module selects one of the Bluetooth or Wi-Fi communications for use based on a type of the one or more sensors in use.

29. The system of claim 27, wherein determining the quality of service (QoS) measurement of the wireless communication link comprises:
computing transmission delays based on time stamps of the vital sign measurements and time of receipt of the vital sign measurement; and
based on the transmission delays, producing the QoS measurement indicative of reliability and latency of the wireless communication link.

30. The system of claim 27, wherein employing the secondary communication technique between the one or more sensors and the monitor module to replace the wireless communication link comprises:
employing an electromagnetic coupling communication.
